(19) United States
(12) Patent Application Publication
(10) Pub. No.: US 2024/0268637 A1
(43) Pub. Date: Aug. 15, 2024

(54) A DEVICE FOR SUPPORTING PROBE AND/OR ENDOSCOPE PARTS OF MEDICAL DEVICES INTRODUCED THROUGH THE MOUTH OF A PATIENT

(71) Applicant: EchoVice ApS, Odense M (DK)

(72) Inventors: Ajithan Christian, Odense (DK); Kristian Bach Laursen, Odense (DK); Jeppe Olesen, Odense (DK)

(21) Appl. No.: 18/568,664

(22) PCT Filed: Jan. 16, 2023
(86) PCT No.: PCT/EP2023/050801

§ 371 Date: Dec. 8, 2023

(30) Foreign Application Priority Data
Jan. 28, 2022 (DK) PA202200079

(51) Int. Cl. A61B 1/00

(52) U.S. Cl. CPC A61B 1/00154; A61B 1/0016

(57) ABSTRACT

A device for supporting probe and/or endoscope parts of medical devices introduced through the mouth of a patient is shown. The device includes a) a first channel part adapted for fitting into the lumen of a bite block for probe and endoscope equipment or configured as a bite block for probe and endoscope equipment, b) a second channel part with a wall section adapted for moving between a first retracted position to a second extended position; and c) a mechanism adapted for displacing the wall section between the first retracted position and the second extended position.

### BACKGROUND OF THE INVENTION

The present invention relates to medical probes or endoscopes, and more specifically to supporting equipment for use with such medical devices.

Many different medical operations and examinations are performed with probes, such as ultrasonic probes, and endoscopes, such as bronchoscopes or laryngoscopes, introduced through the mouth of a patient. The procedure itself is often highly uncomfortable for the subject. For this reason, it is very common for the subject to be sedated during the procedure. The sedative helps the subject to tolerate the procedure, rather than to oppose it, thus enabling the medical staff to perform the procedure undisturbed. Despite this, the subject will often resist entry of the endoscope by not swallowing as requested and by tensing his or her muscles. Furthermore, involuntary muscular reactions also resist the precise positioning of the probe or endoscope.

As an example, the treatment of leaky heart valves with clips or similar systems depends on the ultrasound images provided. The success of the treatment depends on the ultrasound operator being able to create the best images in the right quality at the right time during the operation. Here, the medical staff are challenged because the esophagus cannot be completely anesthetized during the treatment, even under general anesthesia. The result is that the muscles constantly displace the tube of the ultrasound probe within the esophagus. The ultrasound operators must manually counteract these forces by pushing on the ultrasound probe while controlling the position of the ultrasound probe's head.

In addition, the ultrasound operator must also control the ultrasound apparatus to which the probe is connected, including manipulation of the images shown on the apparatus' monitor. Hence, there is a need to reduce the ultrasound operator's manual tasks.

### SUMMARY OF THE INVENTION

Thus, it is an object of the present invention to provide a simple solution that solves the above-mentioned problems.

The present invention provides a new way of supporting probe and/or endoscope parts of medical devices extending into the mouth of a patient. It is a simple device that makes it possible for one person to handle the probe or endoscope without having to interrupt their work to adjust and reposition the tip of the probe or endoscope. Thereby, the total operating or surveying time can be reduced also reducing the risk of the operation or survey. The device will provide fewer interruptions, less frustration, and better ergonomics to the operator. A first aspect relates to a device for supporting probe and/or endoscope parts of medical devices extending into the mouth of a patient, said device comprising:
a first channel part adapted for fitting into the lumen of a bite block for probe and endoscope equipment or configured as a bite block for probe and endoscope equipment;
a second channel part with a wall section adapted for moving between a first retracted position to a second extended position; and
a mechanism adapted for displacing said wall section between its first retracted position and second extended position.

A second aspect relates to a device for supporting probe and/or endoscope parts of medical devices introduced through the mouth of a patient, said device comprising:
a first channel part adapted for fitting into the lumen of a bite block for probe and endoscope equipment or configured as a bite block for probe and endoscope equipment;
a second channel part with a wall section adapted for moving between a first retracted position to a second extended position; and
a mechanism adapted for displacing said wall section between its first retracted position and second extended position;
wherein said second channel part, when said wall section is in its second extended position, is adapted to prevent a probe and/or endoscope part from being moved through said second channel part while still allowing for rotational motion of said probe and/or endoscope part about a longitudinal axis of said probe and/or endoscope part.

A bite block is a, often single-use, medical device used to keep the mouth open during invasive imaging procedures or endoscopy to prevent the patient from biting the probe or endoscope. Bite blocks are generally characterized by the following structural features. A tubular body whose internal lumen serves as the channel for passage of the probe or endoscope, and whose top and bottom outer surfaces serve as the surfaces upon which the subject's teeth bite. These surfaces are generally flattened. A wall centrally connected to the outer end of the tubular body, shaped such that it should lie comfortably outside and around the subject's mouth. It is this wall that fulfils the double function of providing a general alignment direction to the tubular body and of preventing the bite block from falling into the mouth. The wall is also known as the front plate. Optionally, a band connected to the bite block and used to strap the bite block firmly to the subject. Some bite blocks are made of a relatively softer plastic material than others, thereby making a better fit within the oral cavity, such that a strap or band is superfluous.

The device according to the present invention comprises a first channel part either adapted for fitting into the lumen of a bite block for probe and endoscope equipment or configured as a bite block for probe and endoscope equipment. In the latter embodiment, the first channel part may be releasably attached to the device, such as to a second channel part, or a support plate holding the second channel part. The first channel part may thus be for one-time use, i.e., disposable.

A second channel part is provided with a wall section adapted for moving between a first retracted position to a second extended position. This function allows the user to fix probe and/or endoscope parts of medical devices extending through the second channel part and into the mouth of a patient, thereby preventing it from being displaced. The first and second channel parts are connected, e.g., via a support plate for holding other components of the device, such as the mechanism adapted for displacing the second channel part's wall section between its first retracted position and second extended position.

In one or more embodiments, the first channel part is configured for fitting into the cavity of a bite block for probe and endoscope equipment. The first channel part may comprise two separate insert parts configured for fitting into the cavity of a bite block for probe and endoscope equipment.

In one or more embodiments, the mechanism comprises a motor unit operably connected to the second channel part's displaceable wall section. The motor unit is preferably configured for wireless operation, e.g., configured for being controlled by the device for operating the probe or endoscope. The motor unit may be operably connected to the second channel part's displaceable wall section with different types of mechanisms. One non-limiting example may be via a rack and pinion mechanism. Other types of gear mechanisms may also be possible.

It may be necessary for the operator of the probe or endoscope to rotate or turn the probe or endoscope around its longitudinal axis, e.g., to obtain a better position for their tip. To be able to rotate (around its longitudinal axis) and still fix (in the longitudinal direction) the probe and/or endoscope parts extending through the second channel part and into the mouth of a patient, the device may further comprise a roller or ball bearing adapted for rollably supporting probe and/or endoscope parts passing through the first and/or second channel part.

In one or more embodiments, the roller or ball bearing is adapted to move between a first retracted position to a second extended position together with the second channel part's displaceable wall section.

In one or more embodiments, the roller or ball bearing is mounted to the second channel part's displaceable wall section.

Preferably, the device further comprises a roller bearing comprising a pair of cylindrical tubes or rods extending along the length of said first and/or second channel part.

In one or more embodiments, the device further comprises a roller bearing comprising a pair of cylindrical tubes or rods extending along the length of said second channel part.

In one or more embodiments, the device further comprises a roller or ball bearing at least partly positioned within said second channel part and adapted for rollably supporting probe and/or endoscope parts of medical devices positioned within said second channel part.

In one or more embodiments, the device further comprises a roller or ball bearing at least partly positioned within said first channel part and adapted for rollably supporting probe and/or endoscope parts of medical devices positioned within said first channel part.

In one or more embodiments, the roller or ball bearing at least partly positioned within said second channel part is adapted to move between a first retracted position to a second extended position together with said wall section.

In one or more embodiments, the roller or ball bearing at least partly positioned within said second channel part is mounted to said wall section.

In one or more embodiments, the roller or ball bearing at least partly positioned within said second channel part is integrated into said wall section.

In one or more embodiments, the roller or ball bearing at least partly positioned within said second channel part comprises a pair of cylindrical tubes or rods extending along the length of said second channel part.

In one or more embodiments, the roller or ball bearing at least partly positioned within said first channel part comprises a pair of cylindrical tubes or rods extending along the length of said first channel part.

In one or more embodiments, the first channel part is configured for fitting into the lumen of a bite block for probe and endoscope equipment.

A third aspect relates to a medical device comprising a device according to the present invention.

It should be noted that embodiments and features described in the context of one of the aspects of the present invention also apply to the other aspects of the invention.

### BRIEF DESCRIPTION OF THE FIGURES

FIG. 1 and FIG. 2 show perspective views of a device according to the present invention.

FIG. 3 shows a top view of a device according to the present invention.

FIG. 4 shows a cross-sectional view of a device according to the present invention.

FIG. 5 shows a perspective view of a part of a device according to the present invention.

FIG. 6 shows a perspective view of a bite block.

### DETAILED DESCRIPTION OF THE INVENTION

The following examples are not meant to be limiting for the scope of the invention but are merely present to show possible and preferred embodiments of the present invention.

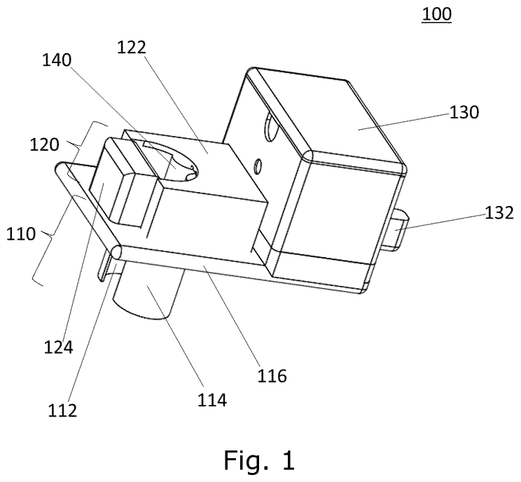

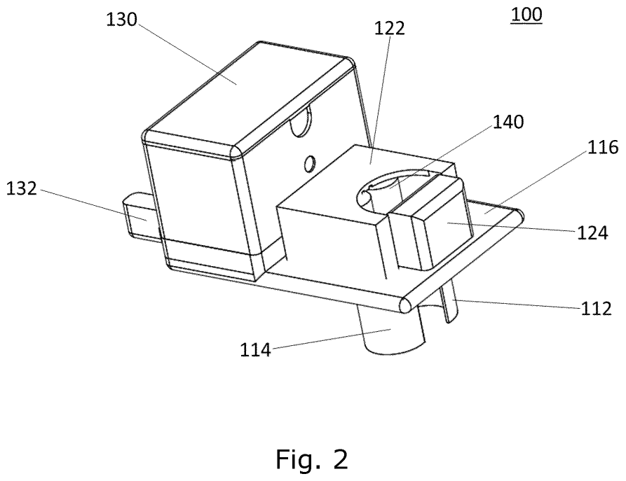

FIG. 1 shows a perspective view of a device 100 in accordance with various embodiments of the present invention. The device 100 comprises a first channel part 110, and a second channel part 120.

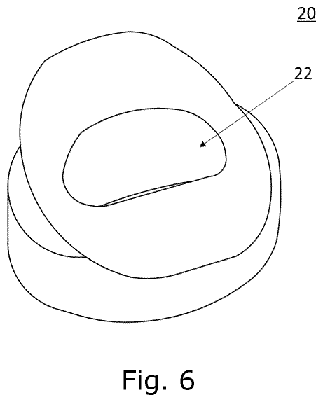

The first channel part 110 is adapted for fitting into the lumen 22 of a bite block 20 (see FIG. 6) for probe and endoscope equipment. The first channel part 110 is shown comprising two separate insert parts 112, 114 configured for fitting into the lumen 22 of a bite block 20 for probe and endoscope equipment. This embodiment ensures a great fit against the lumen 22 wall of a bite block 20 without taking too much space such that the probe or endoscope equipment can move unhindered therethrough.

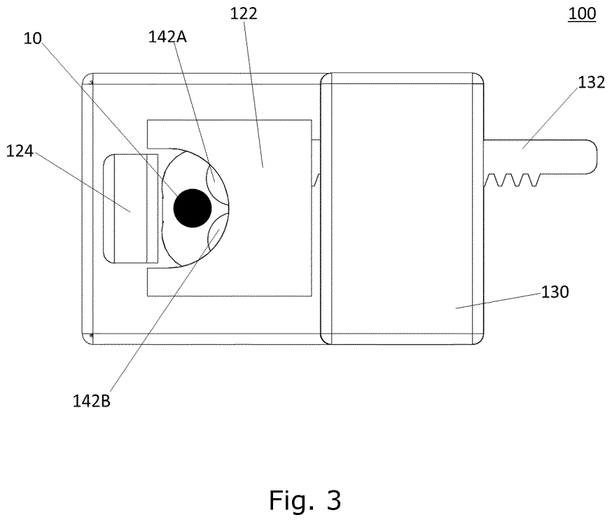

The second channel part 120 with a wall section 122 is adapted for moving between a first retracted position to a second extended position. This movement makes it possible to adjust the width of the second channel part's lumen, thereby fixing the probe or endoscope equipment 10 therein, preferably only in the longitudinal direction, while rotation is still possible. Here, the displaceable wall section 122 is moved back and forth relative to a fixed wall section 124. FIG. 3 shows the wall section 122 in its first retracted position, thereby allowing the probe or endoscope equipment 10 to move freely, e.g., during insertion of the probe or endoscope equipment 10 into the user's trachea or pharynx. The first channel part 110 is shown comprising a support plate 116, adapted for supporting the second channel part's displaceable wall section 122 during its back-and-forth movement. The second channel part's fixed wall section 124 is also mounted thereon.

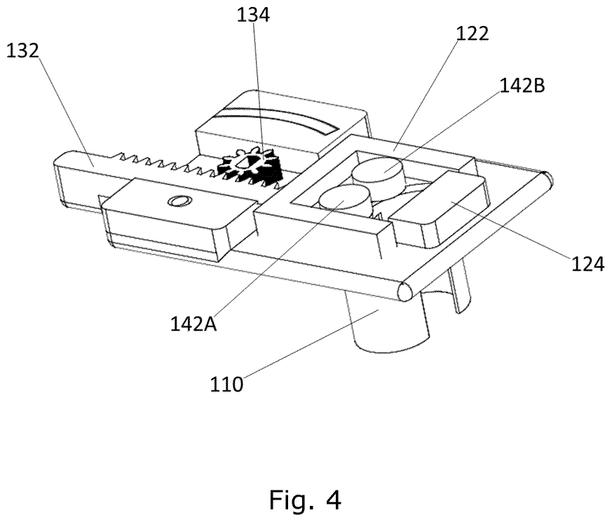

The mechanism adapted for displacing the second channel part's displaceable wall section 122 between its first retracted position and second extended position is here shown comprising a motor unit 130 operably connected to said wall section 122 via a rack 132 and pinion 134 mechanism (see FIG. 4).

To allow the probe or endoscope equipment 10 to rotate freely while being fixed in its longitudinal movement (movement up and down within the subject), the device 100 is shown further comprising a roller bearing 140 adapted for rollably supporting probe or endoscope parts of medical devices positioned within the second channel part 120. The roller bearing 140 may generally be mounted to either one of the second channel part's displaceable 122 or fixed 124 wall section and may comprise two or more cylindrical tubes or rods extending along the length of the second channel part 120, e.g., one or more of the cylindrical tubes or rods mounted on the second channel part's displaceable wall section 122, one or more of the cylindrical tubes or rods mounted on the second channel part's fixed wall section 124, or both. In the shown embodiment, the roller bearing 140 in the form of a pair of cylindrical tubes 142A, 142B (see FIGS. 3-5) are mounted on the second channel part's displaceable wall section 122, thereby making the roller bearing 140 adapted to move between a first retracted position to a second extended position together with said wall section 122.

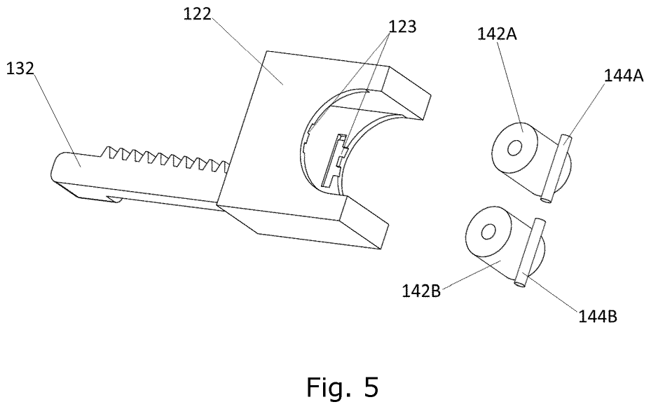

The pair of cylindrical tubes 142A, 142B are mounted to the second channel part's displaceable wall section 122 via a respective pin 144A, 144B (see FIG. 5) fixed with each end in a respective pin mounting recess 123. The pins 144A, 144B allow the cylindrical tubes 142A, 142B to rotate freely relative to the second channel part's displaceable wall section 122.

### REFERENCES

10 Probe and/or endoscope part
20 Bite block
22 Lumen
100 Device
110 First channel part
112 Insert part
114 Insert part
116 Support plate
120 Second channel part
122 Wall section
123 Pin mounting recess
124 Fixed wall section
130 Motor unit
132 Rack
134 Pinion
140 Roller bearing
142 Cylindrical tube
144 Pin

1.-14. (canceled)

15. A device (100) for supporting probe and/or endoscope parts (10) of medical devices introduced through the mouth of a patient, said device (100) comprising:
a first channel part (110) adapted for fitting into the lumen of a bite block for probe and endoscope equipment or configured as a bite block for probe and endoscope equipment;
a second channel part (120) with a displaceable wall section (122) and a fixed wall section (124), the displaceable wall section (122) adapted for moving between a first retracted position to a second extended position, relative to said fixed wall section (124); and
a mechanism adapted for displacing said displaceable wall section (122) between its first retracted position and second extended position;
wherein said second channel part (120), when said displaceable wall section (122) is in its second extended position, is adapted to prevent a probe and/or endoscope part from being moved through said second channel part (120) while still allowing for rotational motion of said probe and/or endoscope part about a longitudinal axis of said probe and/or endoscope part; wherein said mechanism comprises a motor unit (130) operably connected to said displaceable wall section (122).

16. The device according to claim 15, further comprising a roller (140) or ball bearing at least partly positioned within said second channel part (120) and adapted for rollably supporting probe and/or endoscope parts of medical devices positioned within said second channel part (120).

17. The device according to claim 15, further comprising a roller or ball bearing at least partly positioned within said first channel part (110) and adapted for rollably supporting probe and/or endoscope parts of medical devices positioned within said first channel part (110).

18. The device according to claim 15, wherein said motor unit (130) is configured for wireless operation.

19. The device according to claim 15, wherein said motor unit (130) is operably connected to said displaceable wall section (122) via a rack (132) and pinion (134) mechanism.

20. The device according to claim 15, further comprising a roller (140) or ball bearing at least partly positioned within said second channel part (120) and adapted for rollably supporting probe and/or endoscope parts of medical devices positioned within said second channel part (120); wherein said roller (140) or ball bearing at least partly positioned within said second channel part (120) comprises a pair of cylindrical tubes or rods (142A, 142B) extending along the length of said second (120) channel part.

21. The device according to claim 15, further comprising a roller or ball bearing at least partly positioned within said first channel part (110) and adapted for rollably supporting probe and/or endoscope parts of medical devices positioned within said first channel part (110); wherein said roller or ball bearing at least partly positioned within said first channel part (110) comprises a pair of cylindrical tubes or rods extending along the length of said first (110) channel part.

22. The device according to claim 15, wherein said first channel part (110) is configured for fitting into the lumen (22) of a bite block (20) for probe and endoscope equipment.

23. The device according to claim 15, wherein said first channel part (110) is configured for fitting into the lumen (22) of a bite block (20) for probe and endoscope equipment, and wherein said first channel part (110) comprises two separate insert parts (112, 114) configured for fitting into the lumen of a bite block for probe and endoscope equipment.

24. The device according to claim 15, wherein the motor unit is configured for being wirelessly controlled by a device for operating the probe or endoscope.

25. The device according to claim 15, wherein the first channel part (110) is configured as a bite block for probe and endoscope equipment.

26. A medical device comprising:
a bite block (20) adapted for receiving probe and endoscope equipment, said bite block (20) comprising a lumen (22); and
a device (100) for supporting probe and/or endoscope parts (10) of medical devices introduced through the mouth of a patient, said device (100) comprising:
a) a first channel part (110) adapted for fitting into the lumen of a bite block for probe and endoscope equipment or configured as a bite block for probe and endoscope equipment;
b) a second channel part (120) with a displaceable wall section (122) and a fixed wall section (124), the displaceable wall section (122) adapted for moving between a first retracted position to a second extended position, relative to said fixed wall section (124); and
c) a mechanism adapted for displacing said displaceable wall section (122) between its first retracted position and second extended position;
wherein said second channel part (120), when said displaceable wall section (122) is in its second extended position, is adapted to prevent a probe and/or endoscope part from being moved through said second channel part (120) while still allowing for rotational motion of said probe and/or endoscope part about a longitudinal axis of said probe and/or endoscope part; wherein said mechanism comprises a motor unit (130) operably connected to said displaceable wall section (122);
wherein said first channel part (110) is configured for fitting into the lumen (22) of a bite block (20) for probe and endoscope equipment.

27. The medical device according to claim 26, wherein said first channel part (110) comprises two separate insert parts (112, 114) configured for fitting into the lumen of a bite block for probe and endoscope equipment.

28. The medical device according to claim 26, wherein the motor unit is configured for being wirelessly controlled by the device for operating the probe or endoscope.

29. The medical device according to claim 26, further comprising the probe and endoscope equipment.
